(19) United States
(12) Patent Application Publication
(10) Pub. No.: US 2022/0218409 A1
(43) Pub. Date: Jul. 14, 2022

(54) SURGICAL DEVICE WITH SEGMENTED END EFFECTOR

(71) Applicant: GYRUS ACMI, INC. D/B/A OLYMPUS SURGICAL TECHNOLOGIES AMERICA, Westborough, MA (US)

(72) Inventor: Thomas J. Holman, Princeton, MN (US)

(21) Appl. No.: 17/647,047

(22) Filed: Jan. 5, 2022

Related U.S. Application Data
(60) Provisional application No. 63/136,292, filed on Jan. 12, 2021.

(51) Int. Cl. A61B 18/14

(52) U.S. Cl. CPC A61B 18/1445; A61B 2017/2933

(57) ABSTRACT

A surgical device and associated methods such as an end effector are disclosed. The end effector can include: a first component forming a body of the end effector, wherein the first component is formed of a first electrically non-conductive material; and a second component coupled to the first component at a joint, wherein the second component is formed of a second material or is formed of the first material but is processed differently from the first material, wherein the second component connects the first component to the surgical device and has one or more features that are configured to facilitate articulating movement of the first component.

### CROSS-REFERENCE TO RELATED APPLICATION

This application claims the benefit of priority to U.S. Provisional Patent Application Ser. No. 63/136,292, filed Jan. 12, 2021, the contents of which are incorporated herein by reference in their entirety.

### TECHNICAL FIELD

Embodiments described herein generally relate to end effectors for surgical devices. Specific examples of such end effectors include, but are not limited to, a forceps.

### BACKGROUND

Surgical devices for diagnosis and treatment, such as forceps, are often used for medical procedures such as laparoscopic and open surgeries. Forceps can be used to manipulate, engage, grasp, or otherwise affect an anatomical feature, such as a vessel or other tissue of a patient during the procedure. Forceps often include an end effector that is manipulatable from a handle of the forceps. For example, jaws located at a distal end of a forceps can be actuated via elements of the handle between open and closed positions to thereby engage the vessel or other tissue. Forceps can include an extendable and retractable blade that can be extended distally between a pair of jaws to lacerate the tissue. The handle can also be capable of supplying an input energy, such as electromagnetic energy or ultrasound, to the end effector for sealing of a vessel or tissue during a procedure. Improved forceps and other surgical devices are desired.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a side view of an electrosurgical forceps in accordance with an example of the present disclosure.

FIG. 2A illustrates an isometric view of an end effector of the forceps articulated to a closed position.

FIG. 2B illustrates an isometric view of the end effector of the forceps articulated to a partially open position.

FIG. 2C illustrates an isometric view of the end effector of the forceps articulated to an open position.

FIG. 3 is a partially exploded view of the end effector of FIGS. 2A-2C.

FIG. 4A is a perspective view of a body of the end effector having a recess forming a portion of a joint between the body and a frame in accordance with an example of the present disclosure.

FIG. 4B is a partial cross-sectional view of the body through the recess and further illustrating a track that forms part of the recess.

FIG. 5 is the partial cross-sectional view of FIG. 4B but further illustrating the frame received in the recess to form the joint.

FIG. 6 illustrates an exemplary plot of an actuator displacement v. jaw displacement having a non-linear (curved) relationship for the end effector of the forceps of FIGS. 1-5.

FIG. 7 is a partial cross-sectional view through the body illustrating another example of a joint.

FIG. 8 is a perspective view of another example of the body with the recess forming a portion of a joint.

FIG. 8A is a cross-sectional view through a track that forms part of the recess of FIG. 8 showing a curvature of the track in a medial-lateral direction.

FIG. 9 is a perspective view of yet another example of the body and further illustrating a tab for fixating the frame to the body according to an example of the present application.

FIG. 10 shows a flow diagram of a method of manufacture of a forceps in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

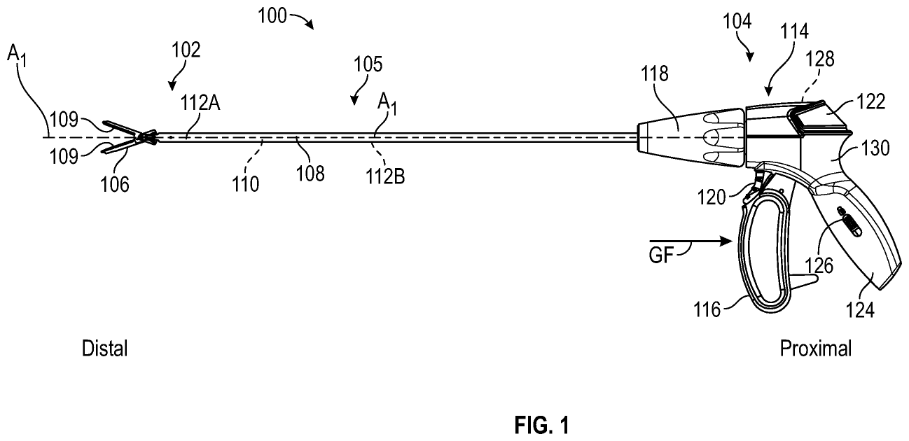

The following disclosure may be used with a number of different types of surgical devices such as tweezers, wound closure devices, etc. One example for illustration shown in FIG. 1 is an electrosurgical forceps.

Electrosurgical forceps can use an articulating jaw to manipulate, engage, grasp, or otherwise affect an anatomical feature, such as a vessel or other tissue of the patient during the procedure. The jaw can include a frame and a body. Typically, the frame and body are constructed of a same material and/or as a single piece construct. However, the body and the frame service different purposes and are subject to different forces. For example, is desirable that the body have an electrically nonconductive property that isolates the electrode from the frame or from another electrode of the opposing jaw to prevent inadvertent shorting from the electrode. The body should also have a stiffness and strength sufficient so that closure force or pressure can be applied by the jaws to the captured anatomical feature.

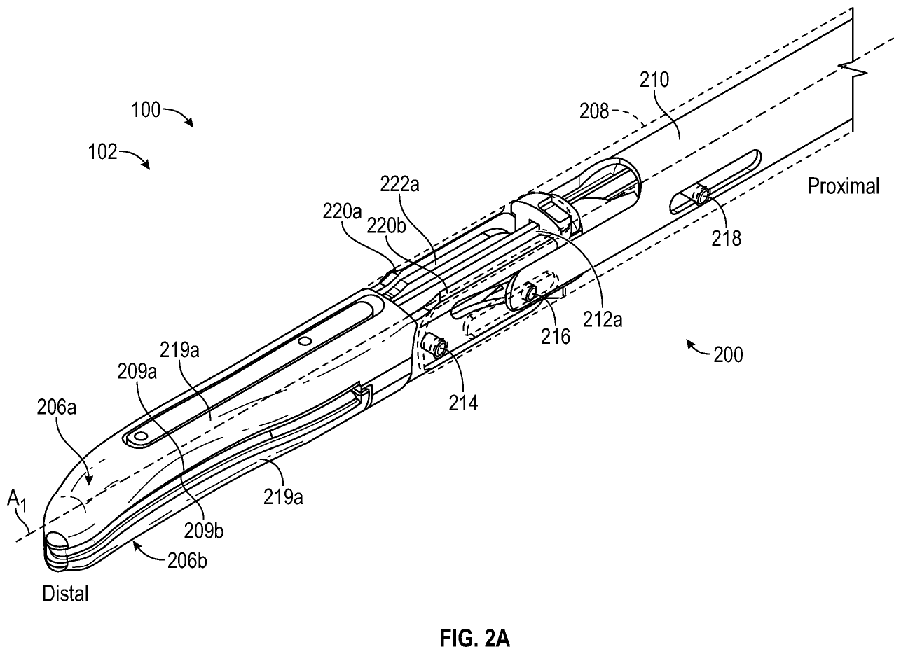

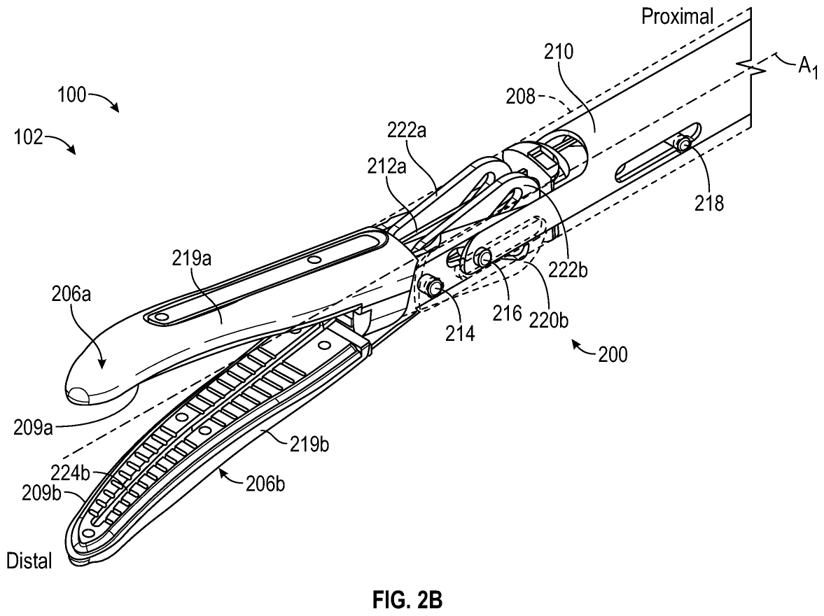

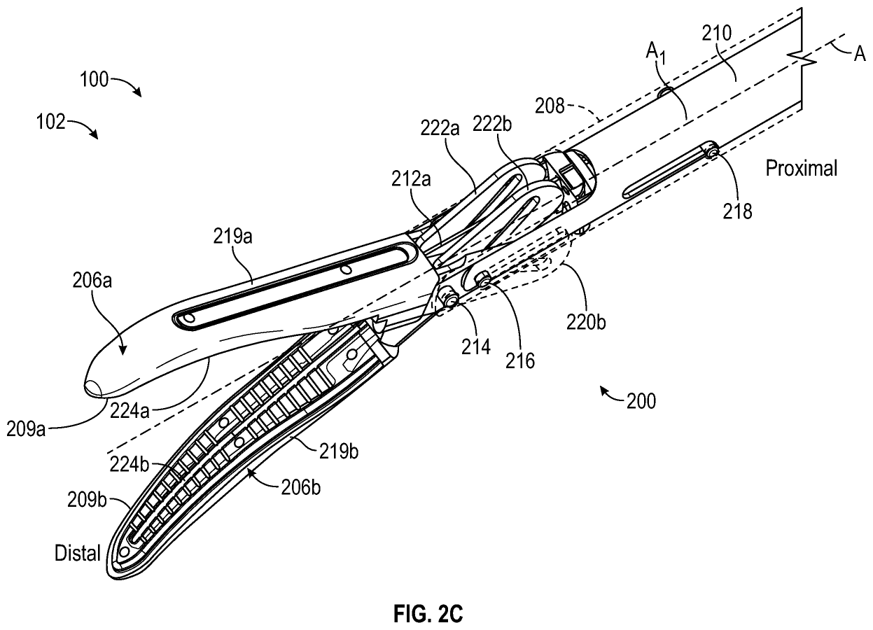

The frame can support the structural loads related to mounting the body to the forceps and for articulating the jaw from the open position of FIGS. 1 and 2C toward or to the closed position of FIG. 2A to capture the anatomical feature. To accomplish this, the frame can have one or more features (e.g., a through hole to support a pivot pin and slots to interact with a reciprocating camming pin). Considering the above, the strength, toughness, and manufacturability requirements of the frame may not align with those of the body.

The present disclosure can help to address these and other issues by using different materials (or a same material that is differently processed) for the body and the frame. Additionally, the present disclosure contemplates the frame can be separated from the body with a joint therebetween. This arrangement allows the manufacturing characteristics and physical properties provided by the material(s) to be selected considering operating criteria.

Furthermore, and regardless of the materials for the body and the frame, the present disclosure contemplates that the joint can be tailored to provide a desired amount of force to deflection between the body and the frame as further discussed herein. This can allow the jaws to be actuated to capture, grasp and manipulate the anatomical feature in a tailored manner. Put another way, the joint can allow a grip force used to actuate the jaws to be tailored with a domain of relatively lower grip force per jaw displacement and domain of relatively higher grip force per jaw displacement, for example.

FIG. 1 illustrates a side view of a forceps 100 showing jaws in an open position. The forceps 100 can include an end effector 102, a handpiece 104, and an intermediate portion 105. The end effector 102 can include jaws 106 (including electrodes 109). In one example, the shaft 108 includes, an inner shaft and an outer shaft, and a blade assembly, although the invention is not so limited. The handpiece 104 can include a housing 114, a lever 116, a rotational actuator 118, a trigger 120, an activation button 122, a handle 124, and a locking mechanism 126. FIG. 1 shows orientation indicators Proximal and Distal and a longitudinal axis A1.

Generally, the handpiece 104 can be located at a proximal end of the forceps 100 and the end effector 102 can be located at the distal end of the forceps 100. The intermediate portion 105 can extend between the handpiece 104 and the end effector 102 to operably couple the handpiece 104 to the end effector 102. Various movements of the end effector 102 can be controlled by one or more actuation systems of the handpiece 104. For example, the end effector 102 can be rotated about the longitudinal axis A1 of the forceps 100. Also, the handpiece 104 can operate the jaws 106, such as by moving the jaws 106 between open and closed position. The handpiece 104 can also be used to operate a cutting blade (not shown) for cutting tissue. The handpiece 104 can also be used to operate the electrode 109 for applying electromagnetic energy to tissue. The end effector 102, or a portion of the end effector 102 can be one or more of: opened, closed, rotated, extended, retracted, and electromagnetically energized.

The housing 114 can be a frame that provides structural support between components of the forceps 100. The housing 114 is shown as housing at least a portion of the actuation systems associated with the handpiece 104 for actuating the end effector 102. However, some or all of the actuation components need not necessarily be contained within the housing 114.

The shaft 108 can include a drive shaft 110 and an outer shaft. The drive shaft 110 can extend through the housing 114 and out of a distal end of the housing 114, or distally beyond housing 114. The jaws 106 can be connected to a distal end of the drive shaft 110. The outer shaft can be a hollow tube positioned around the drive shaft 110. A distal end of the outer shaft can be located adjacent the jaws 106. A blade shaft can also reside within the shaft 108.

A proximal portion of the trigger 120 can be connected to the blade shaft within the housing 114. A distal portion of the trigger 120 can extend outside of the housing 114 adjacent, and in some examples, nested with the lever 116 in the default or unactuated positions. The activation button 122 can be coupled to the housing 114 and can include or be connected to electronic circuitry within the housing 114. Such circuitry can send or transmit electromagnetic energy through the shaft 108 to the electrodes 109. In some examples, the electronic circuitry may reside outside the housing 114 but may be operably coupled to the housing 114 and the end effector 102.

In operation of the forceps 100, a user can grip and use a grip force GF to displace the lever 116 proximally to drive the jaws 106 with an articulating movement from an open position to or toward a closed position. This articulating movement of the jaws 106 can allow the jaws 106 to clamp down on and compress a tissue or other anatomical feature. The handpiece 104 can also allow a user to move the rotational actuator 118 to cause the end effector 102 to rotate, such as by rotating the shaft 108, or inner components associated with the shaft 108. Although described herein with the example of articulating movement, it is contemplated in various embodiments that the term “movement” of the jaws 106 or other components can include: linear movement (e.g., sliding), non-linear movement, constrained linear movement, constrained nonlinear movement, reciprocal movement, oscillating movement, or a combination of articulating movement with any of the linear movement, non-linear movement, constrained linear movement, constrained nonlinear movement, reciprocal movement, oscillating movement or the like.

In some examples, with the tissue compressed, a user can depress the activation button 122 to cause electromagnetic energy, or in some examples, ultrasound, to be delivered to one or more components of the end effector 102, such as electrodes 109 and in turn to a tissue. Application of such energy can be used to seal or otherwise affect the tissue. In some examples, the electromagnetic energy can cause tissue to be coagulated, sealed, ablated, or can cause controlled necrosis.

In some examples, the handpiece 104 can enable a user to extend and retract a blade (not shown), which can be attached to a distal end of a blade shaft. In some examples, the blade shaft can extend an entirety of a length between the handle 104 and the end effector 102. The blade can be extended by displacing the trigger 120 proximally and the blade can be retracted by allowing the trigger 120 to return distally to a default position.

The forceps 100 can be used to perform a treatment on a patient, such as a surgical procedure. In one example, a distal portion of the forceps 100, including the jaws 106, can be inserted into a body of a patient, such as through an incision or another anatomical feature of the patient's body. While a proximal portion of the forceps 100, including housing 114 remains outside the incision or another anatomical feature of the body. Actuation of the lever 116 causes the jaws 106 to clamp onto a tissue. The rotational actuator 118 can be rotated via a user input to rotate the jaws 106 for maneuvering the jaws 106 at any time during the procedure. Activation button 122 can be actuated to provide electrical energy to jaws 106 to cauterize or seal the tissue within closed jaws 106. Trigger 120 can be moved to translate a blade assembly distally in order to cut tissue within the jaws 106.

In some examples, the forceps 100, or other surgical device, may not include all the features described or may include additional features and functions, and the operations may be performed in any order. The handpiece 104 can be used with a variety of other end effectors to perform other methods.

FIG. 2A illustrates an isometric view of the distal portion of the forceps 100 in a closed position. FIG. 2B illustrates an isometric view of the distal portion of the forceps 100 in a partially open position. FIG. 2C illustrates an isometric view of the distal portion of the forceps 100 in an open position. FIGS. 2A-2C are discussed below concurrently.

The forceps 100 can include the end effector 102 that can be connected to a handle (such as the handle 104 illustrated and discussed previously). The end effector 102 will now be discussed and illustrated in greater detail with the use of new reference numbers. The end effector 102 can include jaws 206a and 206b, an outer shaft 208, grip plates 209a and 209b, an inner shaft 210, a blade assembly 212, a pivot pin 214, a drive pin 216, and a guide pin 218. The jaw 206a can include a body 219a and frames 220a and 220b, and the jaw 206b can include a body 219b and frames 222a and 222b. The grip plate 209a can include a blade slot 224a and the grip plate 209b can include a blade slot 224b. The blade assembly 212 can include a blade 212a and a shaft 212b. FIGS. 2A-2C also show orientation indicators Proximal and Distal and a longitudinal axis A1.

The jaws 206a and 206b, in particular, the body 219a and the body 219b can be rigid or semi-rigid members configured to engage tissue. The jaws 206a and 206b can be coupled to the outer shaft 208, such as pivotably coupled, via the frames 220a, 220b, 222a and 222b and the pivot pin 214. The pivot pin 214 can extend through the frames 220a, 220b, 222a and 222b of the jaws 206a and 206b (such as a bore of each of the frames 220a, 220b, 222a and 222b) such that the pivot pin 214 can be received by outer arms of the outer shaft 208. In other examples, the frames 220a, 220b, 222a and 222b can have bosses or other feature to facilitate connection. Thus, the jaws 206a and 206b can be pivotably coupled to the outer shaft 208 via a boss or bosses of the outer shaft 208. In another example, the jaws 206a and 206b can include a boss (or bosses) receivable in bores of the outer shaft 208 to pivotably couple the jaws 206a and 206b to the outer shaft 208. In another example, outer shaft 208 can include a boss (or bosses) receivable in bores of the jaws 206a and 206b to pivotably couple the jaws 206a and 206b to the outer shaft 208.

The frames 220a and 220b (which can be a single frame or a set of frames, that is, two frames, three frames, etc.) can be rigid or semi-rigid members such as flanges located at a proximal portion of the jaw 206a. Similarly, the frames 222a and 222b can be rigid or semi-rigid members such as flanges located at a proximal portion of the jaw 206b. In some examples, the frames 220a and 220b can be positioned laterally outward of the inner frames 222a and 222b, respectively. In other examples, the frames 220a and 220b and 222a and 222b can be interlaced.

The grip plates 209a and 209b of the jaws 206a and 206b can each be a rigid or semi-rigid member configured to engage tissue and/or the opposing jaw to grasp tissue, such as during an electrosurgical procedure. The grip plates 209a and 209b can be held in place by the body 219a and 219b, respectively.

One or more of the grip plates 209a and 209b can include one or more of serrations, projections, ridges, or the like configured to increase engagement pressure and friction between the grip plates 209a and 209b and tissue. The frames 220a and 220b of the upper jaw 206a can extend proximally away from the grip plate 209a and 209b, and in some examples, substantially downward when the upper jaw 206a is in the open and partially open positions (as shown in FIGS. 2B and 2C, respectively). Similarly, the frames 222a and 222b of the lower jaw 206b can extend proximally away from the grip plate, and in some examples, substantially upward when the upper jaw 206a is in the open and partially open positions (as shown in FIGS. 2B and 2C, respectively), such that the jaws 206a and 206b and frames 220 and 222 operate to open and close in a scissoring manner. The jaws 206a and 206b can each include an electrode configured to deliver electricity to tissue (optionally through the grip plates 209a and 209b), and a frame supporting the electrode. The blade slots 224a and 224b of the grip plates 209a and 209b can together be configured to receive a blade between the jaws 206a and 206b, when the jaws are moved out of the open position. In some examples, only one blade slot may be used.

Each of the inner shaft 210 and the outer shaft 208 can be a rigid or semi-rigid and elongate body having a geometric shape of a cylinder, where the shape of the inner shaft 210 matches the shape of the outer shaft 208. In some examples, the inner shaft 210 and the outer shaft 208 can have other shapes such as an oval prism, a rectangular prism, a hexagonal prism, an octagonal prism, or the like. In some examples, the shape of the inner shaft 210 can be different from the shape of the outer shaft 208.

The inner shaft 210 can extend substantially proximally to distally along the axis A1, which can be a longitudinal axis. In some examples, the axis A1 can be a central axis. Similarly, the outer shaft 208 can extend substantially proximally to distally along the axis A1. In some examples, the axis A1 can be a central axis of one or more of the inner shaft 210 and the outer shaft 208. The inner shaft 210 can include an axial bore extending along the axis A1. The outer shaft 208 can also include an axial bore extending along the axis A1. The inner shaft 210 can have an outer dimension (such as an outer diameter) smaller than an inner diameter of the outer shaft 208 such that the inner shaft 210 can be positioned within the outer shaft 208 and such that the inner shaft 210 can be translatable in the outer shaft 208 along the axis A1. The inner shaft 210 can also be referred to as a drive shaft 210, a cam shaft 210, or an inner tube 210. The outer shaft 208 can also be referred to as an outer tube 208.

The blade 212a can be an elongate cutting member at a distal portion of the blade assembly 212. The blade 212a can include one or more sharpened edges configured to cut or resect tissue or other items. The blade assembly 212 can be located within the outer shaft 208 (and can be located within the inner shaft 210). The blade 212a can also be a translating member or electrosurgical component other than a blade. For example, the translating member here blade 212a can be an advancing electrosurgical electrode configured to cut tissue, such as a blunt electrode, an electrosurgical blade, a needle electrode, or a snare electrode.

The guide 218, the drive pin 216, and the pivot pin 214 can each be a rigid or semi-rigid pin, such as a cylindrical pin. The guide 218, the drive pin 216, and the pivot pin 214 can have other shapes in other examples, such as rectangular, square, oval, or the like. In some examples, the pivot pin 214 can have a size (such as a diameter) that is larger than the drive pin 216, as discussed below in further detail. Each pin can have a smooth surface to help reduce surface friction between the pins and components of the forceps 100, such as between the pivot pin 214 and the outer shaft 208 or the drive pin 216 and the frames 220 and 222. Each of the guide 218, the drive pin 216, and the pivot pin 214 can be other components such as one or more projections, bosses, arms, or the like.

The guide 218 can be omitted in some examples, such that the drive pin 216 and the pivot pin 214 can connect the inner shaft 210 to the outer shaft 208 (such as through the jaws 206).

In operation, the inner shaft 210 can be translated using an actuator (such as the lever 116 of FIG. 1). The inner shaft 210 can translate with respect to the outer shaft 208 to move the drive pin 216. The drive pin 216 can engage the frames 220a, 220b and 222a, 222b to facilitate articulating movement of the frames 220a, 220b and 222a, 222b, and hence, the body 219a and the body 219b, between open and closed positions illustrated. Thus, to close the jaws 206a and 206b, the inner shaft 210 can be translated proximally to proximally translate the drive pin 216, which can cause the drive pin 216 to translate proximally within slots. As the drive pin 216 translates proximally in the outer slots, the drive pin 216 can translate proximally along (such as within) the tracks of the frames 220a and 220b of the upper jaw 206a and along the tracks of the frames 222a and 222b of the lower jaw 206b. Proximal translation of the drive pin 216 can cause the jaws 206a and 206b to having articulating movement to close in a scissor type movement.

Although the jaws 206a and 206b are illustrated as having articulating movement between open and closed positions, it is understood that according to further embodiments only one of the jaws 206a or 206b may be moveable while the other can be stationary (i.e. the jaws can have unilateral implementation rather than bilateral implementation). Furthermore, the jaws 206a and/or 206b may not fully achieve the closed position due to engagement with tissue. As discussed above, a single frame can be utilized in alternative to the set of frames illustrated.

The components of the forceps 100 can each be comprised of materials such as one or more of metals, plastics, foams, elastomers, ceramics, composites, combinations thereof, or the like. Materials of some components of the forceps 100 are discussed below in further detail.

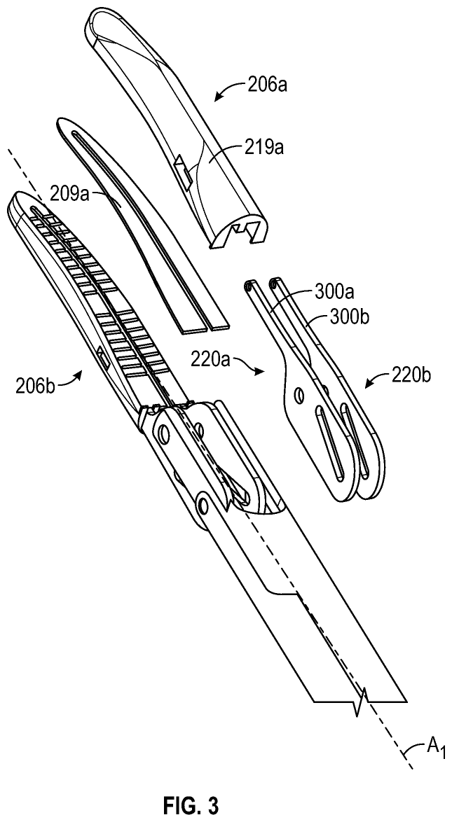

FIG. 3 shows a semi-exploded view of the jaws 206a and 206b from a proximal position. The jaw 206a has been exploded to further illustrate the grip plate 209a, the body 219a and the frame 220a. The frame 220a can include a beam 300a (also referred to as a strut or arm herein). The frame 220b can include a beam 300b (again, also referred to as a strut or arm herein).

The beams 300a and 300b can extend distal from a flange portion of the frames 220a and 220b and can be configured to be received by the body 219a as further described herein. The beams 300a and 300b can form a joint that connects the frames 220a and 220b to the body 219a as further illustrated and described.

According to one example the body 219a (and/or body 219b) can comprise a first material that differs from a second material of the frames 220a and 220b (and/or frames 222a and 222b). According to another example, the body 219a (and/or body 219b) and the frame(s) 220a and 220b (and/or frame(s) 222a and 222b) can comprise a same material but can be processed in a different manner so as to have a different modulus of elasticity, tensile strength or other desired property or characteristic. For example, the body 219a (and/or body 219b) and the frames 220a and 220b (and/or frames 222a and 222b) can both be a same material such as a ceramic. However, the frames 220a and 220b (and/or frames 222a and 222b) can be subject to hot isostatic pressing (HIP) or other processing that differs from the processing of the body 219a (and/or body 219b). This can give the frames 220a and 220b (and/or frames 222a and 222b) different characteristics and properties (e.g., different modulus of elasticity, tensile strength, etc.) from that of the body 219a (and/or body 219b).

According to one embodiment, the first material for the body 219a (and/or body 219b) can be electrically non-conductive according to one embodiment. The electrically non-conductive material can be a polymer a ceramic, a composite, combinations thereof, or the like.

The second material for the frames 220a and 220b (and/or frames 222a and 222b) can be electrically conductive. According to one example, the second material can have a crystalline microstructure or other desired microstructure. The second material can be a metal, metal alloy, a coated metal or metal alloy, a graphite, a carbon, a ceramic, a polymer, a composite, combinations thereof, or the like. Suitable metal and/or metal alloys include Elgiloy® (a non-magnetic Cobalt-Chromium-Nickel-Molybdenum alloy), stainless steel and titanium, for example. Elgiloy® can comprise the second material according to some examples as Elgiloy® has a desirable high modulus of elasticity and a high ultimate tensile strength but can also be subject to a degree of flexure that results from actuation and engagement with anatomical features. Further examples of potential suitable materials are discussed in further detail below.

In one example, the modulus of elasticity of the first material for the body 219a and the second material for the frames 220a and 220b substantially governs how the tool feels when compressing a workpiece. For example, when clamping a tissue during a procedure, the body 219a and frames 220a and 220b of the forceps will flex slightly and provide a clamping force. The amount of flexure is determined by the modulus of elasticity of the material of the body 219a, and also, the modulus of elasticity of the material of the frames 220a and 220b.

It is desirable, when choosing the material for the body 219a and the material of the frames 220a and 220b, to provide a tool feel that a user is expecting and is desirable for the application of the end effector. If a material has too low of a modulus, the tool may not clamp as effectively. In a sense, it may feel too squishy or forgiving. If a material has too high of a modulus, the tool may clamp too severely, and unintentional tissue damage may occur. In a sense, the tool may feel too harsh, and not be forgiving enough to accommodate limited control of application force. It is also desirable for a tool to withstand clamping forces, and not break during use.

As discussed previously, the frames 220a and 220b can be subject to different loading forces, force distribution, deflection, etc. from those the body 219a. It is desirable that the material for the frames 220a and 220b have a high UTS to eliminate plowing in the slot by the drive pin, for example. Furthermore, the beams 300a and 300b of the frames 220a and 220b can be subject to high bending moments when clamping tissue that can result in flexure. Thus, a material able to flex and not fail can also be desirable.

When comparing potential ceramic materials to metals, titanium or stainless steel are good benchmarks. Ranges of yield strength for titanium and titanium alloys are from about 875 MPa to 925 MPa. Ranges of yield strength for stainless steels are from about 200 MPa to 250 MPa. Ranges of modulus of elasticity for titanium and titanium alloys are from about 110 GPa to 120 GPa. Ranges of modulus of elasticity for stainless steel are from about 190 GPa to 200 GPa. Thus, it can be desirable if ceramic or polymer material is selected for the frames and/or body to have properties (yield strength, modulus etc.) in these ranges.

In one example, a ceramic or polymer material can be selected to “feel” like a metal component, with the added advantage of being electrically non-conductive. Selected material can have desired mechanical properties to meet the goal(s) discussed above.

In one example, the body 219a comprises a ceramic. Ceramic materials or electrically non-conductive polymer in surgical tool applications include a number of advantages. One advantage of ceramic materials includes minimal electrical conduction (dielectric behavior) while maintaining desired mechanical properties.

In one example, the body 219a or the frames 220a and 220b can have a sintered ceramic microstructure. This sintered ceramic microstructure can differ from a ceramic microstructure of the other of the body 219a or the frames 220a and 220b as discussed previously. This sintered ceramic microstructure can result from HIP or other processing. Because ceramic is a dielectric, there is no need for separate insulating layers such as a polymer coating, to isolate electrical signals or transmitted energy. A metal body would be coated, or require wires with coated housings to prevent unwanted short circuits.

With a ceramic body (or other non-conducting material as discussed herein), a conducting trace can deposited or otherwise formed directly over a surface of the sintered ceramic microstructure. In one example, one or more of the electrodes is deposited or otherwise formed directly over a surface of the sintered ceramic microstructure. Methods of forming include, but are not limited to, plasma spraying, electrodeposition, chemical deposition, sputtering, or other physical vapor deposition. Depositing an electrode or trace from a vapor, plasma, etc. is easy and inexpensive. When depositing over irregular geometries, it is easy to cover any unusual variations without any undue effort or cost.

In one example, a sintered ceramic microstructure better facilitates the construction of a heat transfer channel without using porosity. In one example, the heat transfer channel includes a thermal conductive trace that is coupled to the sintered ceramic microstructure. Examples of thermal conductive traces include metallic traces. Metallic traces may be deposited or otherwise attached using methods described above, such as plasma spraying, electrodeposition, chemical deposition, sputtering, or other physical vapor deposition. Further details of the forceps construction and other advantages can be found in U.S. Ser. No. 63/032,141, filed on May 29, 2020, entitled “MONOLITHIC CERAMIC SURGICAL DEVICE AND METHOD”, to U.S. Ser. No. 62/826,532, filed on Mar. 29, 2019, entitled “BLADE ASSEMBLY FOR FORCEPS”, to U.S. Ser. No. 62/826,522 filed on Mar. 29, 2019, entitled “SLIDER ASSEMBLY FOR FORCEPS”, to U.S. Ser. No. 62/841,476, filed on May 1, 2019, entitled “FORCEPS WITH CAMMING JAWS”, and to U.S. Ser. No. 62/994,220, filed on Mar. 24, 2020, entitled “FORCEPS DEVICES AND METHODS”, the disclosure of each of which is hereby incorporated by reference herein in its entirety

In one example, the improved ability to construct complex geometries in a green state, then sinter to form a final component better facilitates construction of a heat transfer channel. In one example, the heat transfer channel includes a trench with a metal trace formed within the trench. Such a configuration provides thermal insulation from surrounding tissue or other structures on three sides, with heat conduction being channeled along the metallic trace.

In one example, the body 219a includes yttria stabilized zirconia. In one example, the body 219a includes zirconia toughened alumina. Ranges of modulus of elasticity for yttria stabilized zirconia are from about 200 GPa to 210 GPa. Ranges of modulus of elasticity for zirconia toughened alumina are from about 350 GPa to 370 GPa. Tensile strength for yttria stabilized zirconia is about 500 MPa. Tensile strength for zirconia toughened alumina is about 290 MPa. Although yttria stabilized zirconia and zirconia toughened alumina are used as examples, the invention is not so limited. Other ceramic materials that exhibit dielectric behavior and have elastic moduli similar to metals are also within the scope of the invention.

By choosing a ceramic or polymer material with appropriate mechanical properties, a metal component may be replaced with a ceramic component. In one example, this provides a lower cost option of manufacturing. In one example, this provides more options for complex component geometries. In one example, this provides electrical insulation without the need for a separate insulative coating.

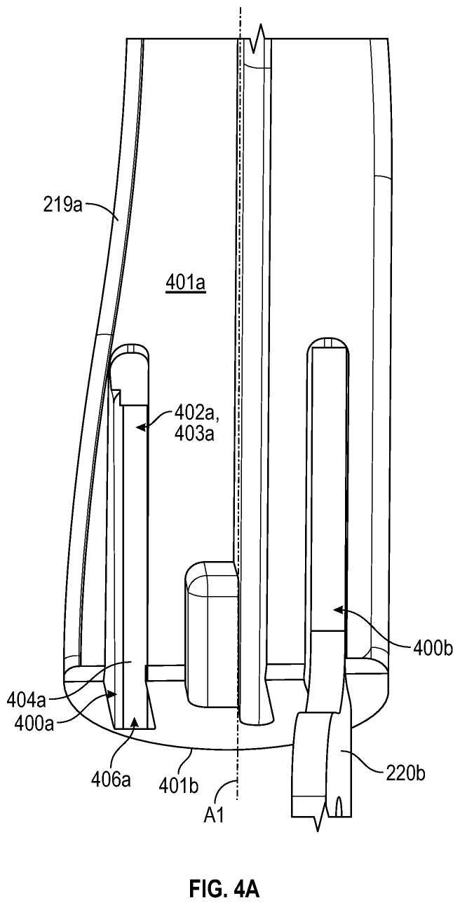

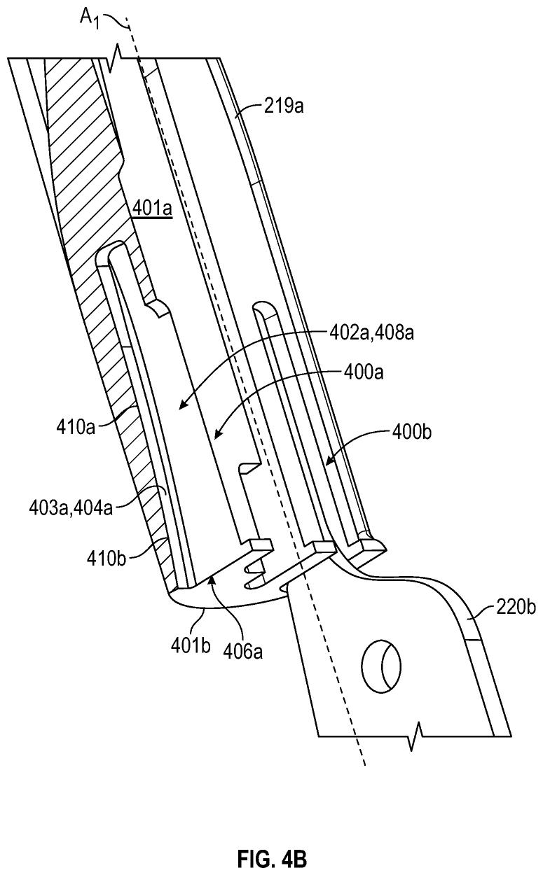

FIG. 4A shows a perspective view of the body 219a with the frame 220a removed to show a part of a joint 400a formed by the body 219a in further detail. FIG. 4B shows the portion of the joint 400a formed by the body 219a via a cross-section through the body 219a. FIGS. 4A and 4B show a joint 400b between the frame 220b and the body 219a, which can be configured in a similar manner as the joint 400a.

The body 219a can include an inward surface 401a and an outward surface 401b. As shown in FIGS. 4A and 4B, the part of the joint 400a can comprise a recess 402a in the body 219a. The recess 402a can be configured to receive the beam 300a of the frame 220a (FIG. 3). The joint 400a can include a track 403a. The track 403a can be formed by one or more surfaces 404a. The one or more surfaces 404a can include outermost surface(s) toward the outward surface 401b that form a bottom of the recess 402a, for example. The track 403a can extend to adjacent a distal end of the recess 402a to a proximal opening 406a to the recess 402a.

As shown in FIG. 4B, the recess 402a can have a second opening 408a along the inward surface 401a of the body 219a. This second opening 408a can be covered by the grip plate 209a (FIG. 3), for example. The grip plate 209a can seat on the inward surface 401a. A distal most portion of the recess 402a can be enclosed on an inward side as shown in FIG. 4B.

The track 403a can be arranged opposing the second opening 408a. As shown in FIG. 4B, the one or more surfaces 404A can be arcuately curved such the joint 400a is tapered from distal to proximal. Put another way, a proximal-most portion of the joint 400a can be relatively larger in cross-section than a distal-most portion of the joint 400a.

The arcuate shape of the track 403a can be formed by one, two or more arcuate segments such as arcuate segments 410a and 410b. Arcuate segments can be continuous or can be separated by other features or surface shapes as discussed further herein. The arcuate segment 410a can be located distal of the arcuate segment 410b and can have a relatively smaller degree of curvature than the arcuate segment 410b as measured distal to proximal and radially along axis A1 (and in the inward/outward radial direction relative to axis A1). The arcuate segment 410b can be located proximal of the arcuate segment 410a and can connect therewith. The arcuate segment 410b can have a relatively greater degree of curvature than the arcuate segment 410a.

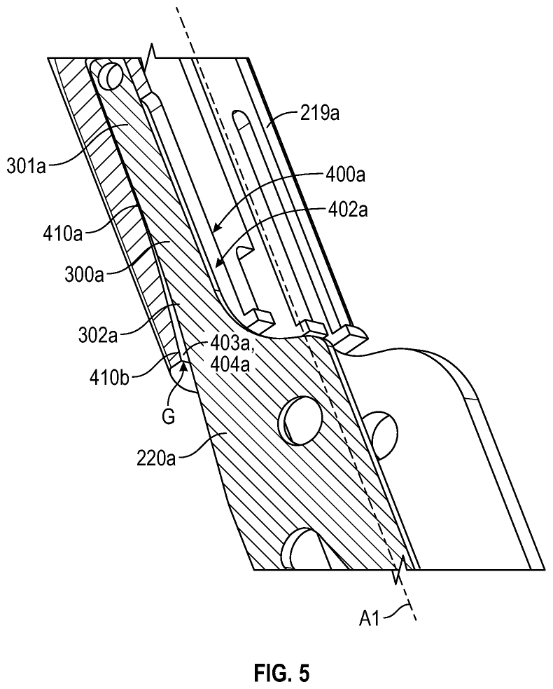

FIG. 5 shows the joint 400a as formed by the body 219a and the frame 220a. Thus, the frame 220a, in particular, the beam 300a, is inserted in the recess 402a. A distal most portion 301a of the beam 300a can be snapped in and captured in intimate contact with one or more surfaces that form the recess 402a. A plug or tab (shown in FIG. 9) may be placed, adhered, or molded into the recess 402a after the beam 300a is snapped into place. Thus, the distal most portion 301a of the beam 300a can be captured in a manner such that it may not be in a pivoting relationship with the body 219a, and in particular, the track 403a.

FIG. 5 shows an arrangement where the distal most portion 301a of the beam 300a of the frame 220a is in intimate contact but a more proximal portion 302a of the beam 300a is able to deflect under applied load. Thus, the shape of the track 403a, in particular with the arcuate segment(s) 410a and/or 410b provide for a gap G between the more proximal portion 302a and the one or more surfaces 404a adjacent the proximal end portion of the joint 400a. This gap G can allow for an amount of flexure (i.e. relative movement) of the more proximal portion 302a of the beam 300a with increased loading until intimate contact between the more proximal portion 302a and the beam 300a is achieved. Put another way, after an amount of articulating, jaw displacement (from the open position toward the closed position) causes the gap G between the more proximal portion 302a and the one or more surfaces 404a can be taken up. Once the gap G is taken up, the load v. deflection relationship of the jaw changes and the joint 400a stiffens.

The joint 400a can allow for an amount of relative movement between the body 219a and the frame 220a during a first regime of closure that imparts a smaller moment upon the body 219a so that a lower force/pressure jaw closure can be utilized than would be the case if the body 219a and the frame 220a were simply in intimate contact for an entirety of the articulating movement of the jaws.

Thus, the joint 400a can be configured to provide for a relative movement between the body 219a and the frame 220a for a first portion of actuation of the body 219a through a first part of articulating movement. Additionally, the joint 400a can be configured to provide for intimate contact between the body 219a and the frame 220a (in particular the more proximal portion 302a) through a second portion of actuation of the body 219a through a second part of the articulating movement.

Furthermore, the joint 400a can be configured to allow a first relative movement between the body 219a and the frame 220a during a first part of articulating movement of the body 219a. The joint 400a can be configured to allow a second more restrictive relative movement of the body 219a relative to the frame 220a during a second part of actuation of the body 219a. This can result from the shape of the track 403a with the two (or more) arcuate segments 410a and 410b. Thus, the joint 400a can have a first shape at a first portion thereof (e.g. as a result of the arcuate segment 410a) such that during a first part of the articulating movement of the body 219a, the body 219a can be subject to a first bending moment (as a result of engaging tissue or other anatomy). The joint 400a can have a second shape at a second portion thereof (e.g. as a result of the arcuate segment 410b) such that during a second part of the articulating movement of the body 219a, the body 219a can be subject to a second different bending moment.

The joint 400a can be configured such that at least two different actuation forces (i.e. two different grip forces GF on lever 116 of FIG. 1) are applied to achieve actuation of the body 219a from the open position toward the closed position. As discussed, the joint 400a can be configured such that the body 219a can be subject to at least two different bending moments during actuation of the body 219a during articulating movement from the open position toward the closed position.

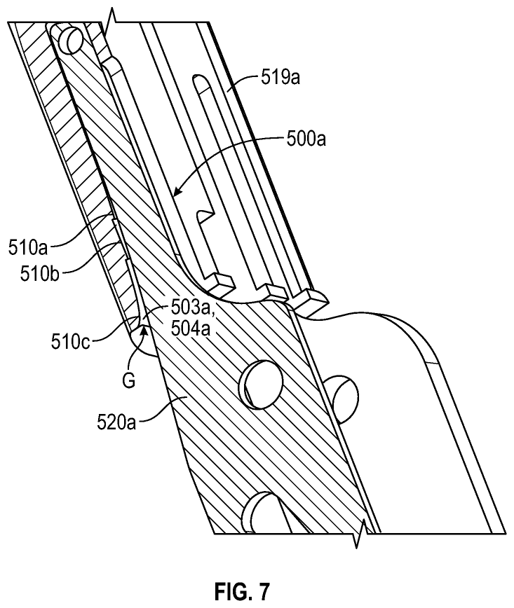

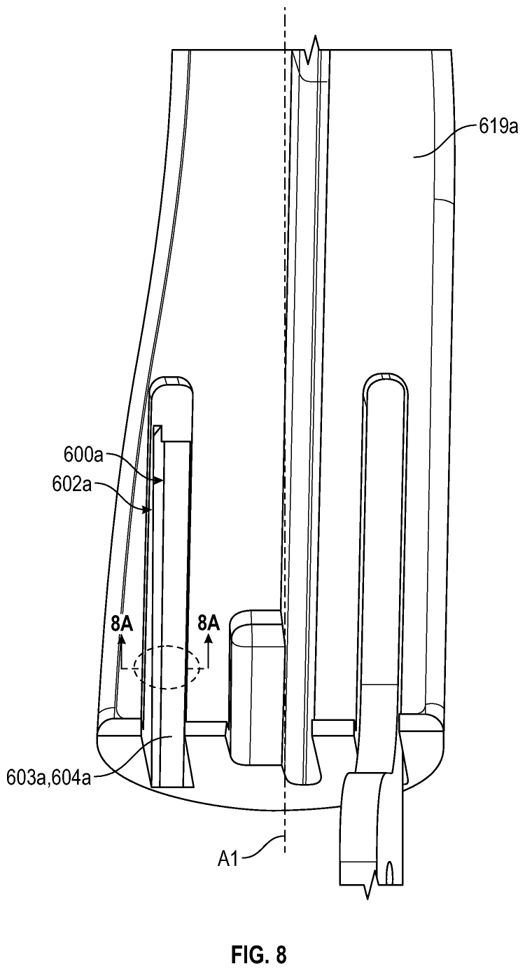

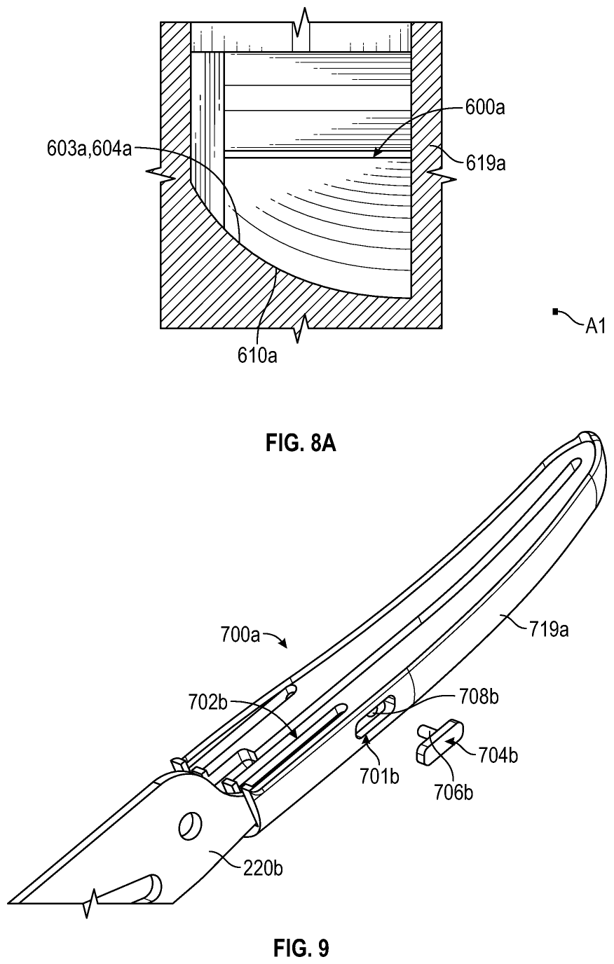

The geometry of the joint 400a can be tailored according to desired closure regimes or other requirements. It is understood that the track 403a need not be arcuate in some examples. For example, the beam 300a along the surface interfacing the track 403a could be arcuate (i.e., could have one or more arcuate segments). Other geometries (e.g., flat, undulating, irregular, complex, mixed, etc.) for the joint 400a (whether for the track 403a and/or the beam 300a) are contemplated and further examples are illustrated in FIGS. 7, 8 and 8A as examples. It is also contemplated that in some cases rather than stiffness of the joint increasing toward closure, the joint can be configured such that stiffness of the joint could decrease toward and/or to closure relative to that near and adjacent the open jaw position.

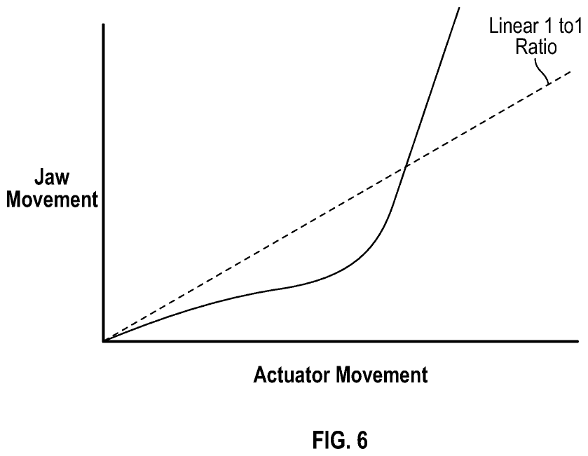

FIG. 6 shows an exemplary plot of an example of jaw displacement v. actuator displacement. The actuator displacement can correlate to displacement of the lever 116 of FIG. 1. As shown in FIG. 6, the jaw displacement does not correlate in a linear manner (linear manner indicated with dashed line) with the actuator displacement. As shown from the plot, an early part of the displacement of the actuator results in relatively less jaw displacement (i.e. the curve has slope of less than 1.0). However, during a latter part of displacement of the actuator, relatively more displacement of the jaw occurs (i.e., the curve has a slope of more than 1.0). As a result of the configuration of the joint, various closure force (i.e. grip force GF of FIG. 1) v. jaw displacement characteristics can tailored as desired. As shown in FIG. 6. the jaw can have at least two closure regimes including a first regime 499a and a second regime 499b. The plot of FIG. 6 illustrates that at least two different bending moments (and two different actuation forces) can be applied through articulating movement of the jaw. Thus, the shape of the joint results in a step function curvature between jaw displacement and actuator displacement. Put another way, the bending moment applied to the frames increases dramatically near closure of the jaws to achieve a same relative articulating displacement of the jaw as compared to the bending moment applied during initial closure of the jaw.

FIG. 7 shows another example of a joint 500a between a body 519a and a frame 520a. The joint 500a differs from that of the joint 400a previously discussed in that a track 503a of the joint 500a along the body 519a includes a first arcuate segment 510a, a feature 510b and a second arcuate segment 510c. The feature 510b can be positioned between the first arcuate segment 510a and the second arcuate segment 510c. The feature 510b can be of any shape as desired. The shape selected can be dependent on the closure regime(s) for the jaws desired. However, in FIG. 7 the feature 510b is illustrated as a non-arcuate (i.e. substantially flat) region of the track 503a designed to create a second region of intimate contact that differs from the first region previously discussed with regard to FIG. 5. This first region of intimate contact remains in the embodiment of FIG. 7 and occurs when no gap remains and the beam and second arcuate segment 510c come into intimate contact. However, in FIG. 7 the first region of intimate contact is spaced from the second region along the track 503a by the second arcuate segment 510c. The second arcuate segment 510c can be thought of as a region of relatively less (or no) intimate contact as compared with the first and second regions. It is contemplated that the feature 510b could be a ridge, bump, tab, mesa or other type of projection extending from the first arcuate segment 510a. The feature 510b could also be a divot or other recess according to other examples. It is understood that the joint 500a provides for different closure regimes than that of joint 400a.

FIG. 8 shows yet another example of a joint 600a between a body 619a and a frame. The frame has been removed in FIG. 8 to further illustrate a recess 602a in the body 619a. The recess 602a can be configured to receive a beam such as the beam 300a of the frame 220a (FIGS. 3 and 5) as previously described. The joint 600a can include a track 603a. The track 603a differs from that of the track 403a previously described.

FIG. 8A shows a cross-section of the track 603a. The track 603a has an arcuate segment 610a along a surface 604a that forms the track 603a. FIG. 8A illustrates that the track 603a can have an arcuate curvature along an axis perpendicular to a longitudinal axis A1 of the jaw. This can counteract an off-axis roll of the jaw upon initial contact with tissue of a patient. Thus, the track 603a can have a curvature along a third direction (medial/lateral relative to the axis A1) in addition to (or in alternative to) one or both of the curvatures discussed previously as measured distal to proximal and radially along axis A1 in the inward/outward radial direction relative to axis A1. Thus, the track 603a and surface 604a, can have at least one of the plurality of arcuate segments that form them curved along an axis perpendicular to a longitudinal axis of the jaw to counteract an off-axis roll of the jaw upon initial contact with tissue of a patient.

FIG. 9 shows another example of a jaws 700a with a body 719a similar to those as described previously. The body 719a includes a lateral opening 701b to a recess 702b. This lateral opening 702b can be configured for fabricating the recess 702b and connecting the body and the frame according to some examples. The lateral opening 702b can be configured to receive a tab 704b. The tab 704b can have a projection 706b designed to be received in an aperture 708b of the frame 220b. The tab 704b can be placed, adhered, or molded into the recess 702b after the beam 300a portion of the frame 220b is snapped into place as previously described.

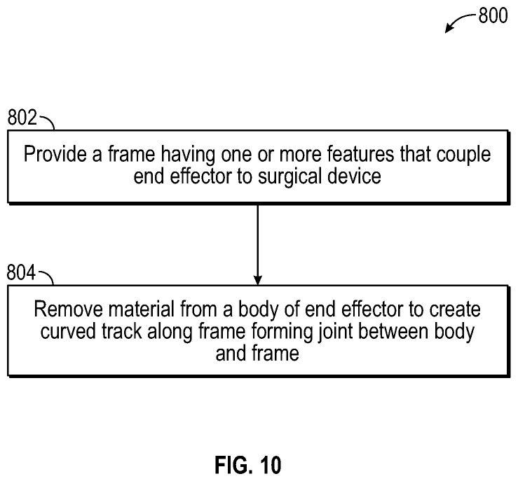

FIG. 10 illustrates a method of forming an end effector for a surgical device according to on example. The method 800 can include providing 802 a frame having one or more features that couple the end effector to the remainder of the surgical device. The one or more features can be configured to facilitate articulating movement of the end effector. The method 800 can include removing 804 material from a body to create a track for the frame. The track can have a plurality of arcuate segments each having a different degree of curvature relative to one another.

The method can further include other steps or features such as shaping the track and frame to provide the body with at least two different bending moments during actuation of the body through the articulating movement. The forming of the body can be of a first electrically non-conductive material. The frame can be formed of a different second material with a crystalline microstructure. The method can include removing material from the body to create the track at least partially through the lateral window (FIG. 9) along a side of the track.

To better illustrate the method and apparatuses disclosed herein, a non-limiting list of embodiments is provided here:

Example 1 is an end effector for a surgical device, optionally comprising: a first component forming a body of the end effector, wherein the first component is formed of a first electrically non-conductive material; and a second component coupled to the first component at a joint, wherein the second component is formed of a second material or is formed of the first material but is processed differently from the first material, wherein the second component connects the first component to the surgical device and has one or more features that are configured to facilitate articulating movement of the first component.

Example 2 is the end effector of Example 1, optionally the first electrically non-conductive material is a ceramic.

Example 3 is the end effector of Example 1, optionally the first electrically non-conductive material is one of a ceramic, a polymer or a composite thereof.

Example 4 is the end effector of any one of Examples 1-3, optionally further comprising an electrode held by the first component, wherein the first electrically non-conductive material isolates the electrode from one or more of a second electrode or the second component.

Example 5 is the end effector of any one of Examples 1-4, wherein the second material is Cobalt-Chromium-Nickel-Molybdenum alloy.

Example 6 is the end effector of any one of Examples 1-4, optionally the second material is at least one of a metal, a metal alloy, a graphite or a carbon.

Example 7 is the end effector of Example 1, optionally both the first component and the second component are both a same ceramic, and wherein the first component has different material properties than the second component.

Example 8 is the end effector of any one of Examples 1-7, optionally the surgical device comprises a forceps, and wherein the first component comprises a jaw body and the second component comprises a frame that facilitates articulating movement of the jaw body.

Example 9 is the end effector of Example 8, optionally the one or more features of the frame comprise one or more of a pivot journal or cam interfacing slot.

Example 10 is the end effector of any one of Examples 1-9, wherein the joint is configured to allow a first relative movement of the first component relative to the second component during a first part of articulating movement of the first component, and wherein the joint is configured to allow a second more restrictive relative movement of the first component relative to the second component during a second part of the articulating movement of the first component.

Example 11 is the end effector of any one of Examples 1-9, optionally the joint has a first shape at a first portion thereof such that, during a first part of the articulating movement of the first component, the first component is subject to a first bending moment, wherein the joint has a second shape at a second portion thereof such that during a second part of the articulating movement of the first component the first component is subject to a second different bending moment.

Example 12 is the end effector of any one of Examples 1-9, optionally the joint is configured to provide for a relative movement between the first component and the second component for a first portion of the articulating movement of the first component, and wherein the joint is configured to provide for intimate contact between the first component and the second component through a second portion of the articulating movement of the first component.

Example 13 is the end effector of any one of Examples 10-12, optionally the joint is configured such that at least two different actuation forces are applied to achieve articulating movement of the first component.

Example 14 is the end effector of any one of Examples 10-12, optionally the joint is configured such that the first component is subject to at least two different bending moments during articulating movement of the first component.

Example 15 is the end effector of Example 14, optionally the joint provides the first component with at least two closure regimes such that a plot of the at least two different bending moments through articulating movement has a step function.

Example 16 is the end effector of any one of Examples 1-15, optionally the joint has a plurality of arcuate segments each having a different degree of curvature relative to one another.

Example 17 is a forceps, optionally comprising: a shaft; an actuator routed along the shaft; and a jaw positioned at an end portion of the shaft and coupled to the actuator, the jaw optionally comprising: a body, an electrode coupled to the body, and a frame coupled to the body at a joint and coupled to the actuator, wherein, when actuated by the actuator, the frame is configured to facilitate articulating movement of the body relative to the shaft, and wherein the joint is shaped such that the actuator applies at least two different forces each of a different degree to the frame through the articulating movement of the body.

Example 18 is the forceps of Example 17, optionally the body is formed of a first electrically non-conductive material that electrically isolates the electrode, and wherein the frame is formed of a different second material with a crystalline microstructure.

Example 19 is the forceps of any one of Examples 17-18, optionally the joint is shaped to provide the body with at least two different bending moments during articulating movement of the body.

Example 20 is the forceps of Example 16, optionally the body has at least two closure regimes such that a plot of the at least two different bending moments during the articulating movement has a step function.

Example 21 is the forceps of any one of Examples 17-20, wherein the joint has a plurality of arcuate segments each having a different degree of curvature relative to one another.

Example 22 is the forceps of Example 21, optionally at least one of the plurality of arcuate segments is curved along an axis perpendicular to a longitudinal axis of the jaw to counteract an off-axis roll of the jaw upon initial contact with tissue of a patient.

Example 23 is the forceps of any one of Examples 17-22, optionally the joint allows relatively more travel of the body per an amount of applied force by the actuator upon initial contact with a tissue of a patient and through a first part of the articulating movement, and wherein the joint allows for relatively less travel of the body with the amount of applied force by the actuator through a second part of the articulating movement of the body.

Example 24 is a method of forming an end effector of a surgical device, optionally comprising: providing a frame having one or more features that couple the end effector to the remainder of the surgical device, the one or more features configured to facilitate articulating movement of the end effector; and removing material from a body to create a track for the frame, wherein the track has a plurality of arcuate segments each having a different degree of curvature relative to one another.

Example 25 is the method of Example 24, optionally further comprising shaping the track and frame to provide the body with at least two different bending moments during articulating movement of the body.

Example 26 is the method of any one of Examples 24-25, optionally further comprising: forming the body of a first electrically non-conductive material; and forming the frame of a different second material with a crystalline microstructure.

Example 27 is the method any one of Examples 24-26, optionally shaping the track and frame to provide for two or more regions of intimate contact therebetween during the articulating movement, wherein the two or more regions of intimate contact are spaced by at least one region of relatively less contact between the track and frame.

Example 28 is the method of Example 27, optionally the track is configured such that a relatively higher grip force is applied for the articulating of movement of the end effector through one of the two or more regions of intimate contact and a relatively lower grip force is applied for the at least one region.

Example 29 is the method of any one of Examples 24-28, optionally removing material from the body to create the track is at least partially performed through one or more windows along a side of the track.

Example 30 is the method of Example 29, optionally further comprising affixing the frame to the body with a tab inserted through the one or more windows.

Example 31 is an end effector for a surgical device, including: a first component forming a body of the end effector, wherein the first component is formed of a first electrically non-conductive material; and a second component coupled to the first component at a joint, wherein the second component is formed of a second material or is formed of the first material but is processed differently from the first material, wherein the second component connects the first component to the surgical device and has one or more features that are configured to facilitate movement of the first component.

Example 32 is the end effector of Example 31, wherein the first electrically non-conductive material is a ceramic.

Example 33 is the end effector of any of Examples 31-32, wherein the first electrically non-conductive material is one of a ceramic, a polymer or a composite thereof.

Example 34 is the end effector of any of Examples 31-33, further including an electrode held by the first component, wherein the first electrically non-conductive material isolates the electrode from one or more of a second electrode or the second component.

Example 35 is the end effector of any of Examples 31-34, wherein the second material is Cobalt-Chromium-Nickel-Molybdenum alloy.

Example 36 is the end effector of any of Examples 31-35, wherein the second material is at least one of a metal, a metal alloy, a graphite or a carbon.

Example 37 is the end effector of any of Examples 31-36, wherein both the first component and the second component are both a same ceramic, and wherein the first component has different material properties than the second component.

Example 38 is the end effector of any of Examples 31-37, wherein the surgical device includes a forceps, wherein the first component includes a jaw body and the second component includes a frame that facilitates an articulating movement of the jaw body, and wherein the one or more features of the frame include one or more of a pivot journal or cam interfacing slot.

Example 39 is the end effector of any of Examples 31-38, wherein the joint is configured to allow a first relative movement of the first component relative to the second component during a first part of articulating movement of the first component, and wherein the joint is configured to allow a second more restrictive relative movement of the first component relative to the second component during a second part of the articulating movement of the first component.

Example 40 is the end effector of any of Examples 31-39, wherein the joint has a first shape at a first portion thereof such that, during a first part of the movement of the first component, the first component is subject to a first bending moment, wherein the joint has a second shape at a second portion thereof such that during a second part of the movement of the first component the first component is subject to a second different bending moment.

Example 41 is the end effector of any of Examples 31-40, wherein the joint is configured to provide for a relative movement between the first component and the second component for a first portion of the movement of the first component, and wherein the joint is configured to provide for intimate contact between the first component and the second component through a second portion of the movement of the first component.

Example 42 is the end effector of any of Examples 31-41, wherein the joint is configured to provide the first component with at least two closure regimes such that a plot of at least two different bending moments through the movement has a step function.

Example 43 is the end effector of any of Examples 31-42, wherein the joint has a plurality of arcuate segments each having a different degree of curvature relative to one another.

Example 44 is a forceps, including: a shaft; an actuator routed along the shaft; and a jaw positioned at an end portion of the shaft and coupled to the actuator, the jaw including: a body, an electrode coupled to the body, and a frame coupled to the body at a joint and coupled to the actuator, wherein, the joint is shaped such that the actuator applies at least two different forces each of a different degree to the frame through a movement of the body.

Example 45 is the forceps of Example 44, wherein the body is formed of a first electrically non-conductive material that electrically isolates the electrode, and wherein the frame is formed of a different second material with a crystalline microstructure.

Example 46 is the forceps of any of Examples 44-45, wherein, when actuated by the actuator, the frame is configured to facilitate articulating movement of the body relative to the shaft, and wherein the joint is shaped to provide the body with at least two different bending moments during articulating movement of the body.

Example 47 is the forceps of any of Examples 44-46, wherein the body has at least two closure regimes such that a plot of the at least two different bending moments during the articulating movement has a step function.

Example 48 is the forceps of any of Examples 44-47, wherein the joint has a plurality of arcuate segments each having a different degree of curvature relative to one another, and wherein at least one of the plurality of arcuate segments is curved along an axis perpendicular to a longitudinal axis of the jaw to counteract an off-axis roll of the jaw upon initial contact with tissue of a patient.

Example 49 is the forceps of any of Examples 44-48, wherein the joint allows relatively more travel of the body per an amount of applied force by the actuator upon initial contact with a tissue of a patient and through a first part of the movement, and wherein the joint allows for relatively less travel of the body with the amount of applied force by the actuator through a second part of the movement of the body.

Example 50 is an end effector for a surgical device, including: a first component forming a body of the end effector; and a second component coupled to the first component at a joint, wherein the second component connects the first component to the surgical device and has one or more features that are configured to facilitate movement of the first component, wherein the joint is shaped such that an actuator applies at least two different forces each of a different degree to the second component through an articulating movement of the body.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. An end effector for a surgical device, comprising:
a first component forming a body of the end effector, wherein the first component is formed of a first electrically non-conductive material; and
a second component coupled to the first component at a joint, wherein the second component is formed of a second material or is formed of the first material but is processed differently from the first material, wherein the second component connects the first component to the surgical device and has one or more features that are configured to facilitate movement of the first component.

2. The end effector of claim 1, wherein the first electrically non-conductive material is a ceramic.

3. The end effector of claim 1, wherein the first electrically non-conductive material is one of a ceramic, a polymer or a composite thereof.

4. The end effector of claim 1, further comprising an electrode held by the first component, wherein the first electrically non-conductive material isolates the electrode from one or more of a second electrode or the second component.

5. The end effector of claim 1, wherein the second material is Cobalt-Chromium-Nickel-Molybdenum alloy.

6. The end effector of claim 1, wherein the second material is at least one of a metal, a metal alloy, a graphite or a carbon.

7. The end effector of claim 1, wherein both the first component and the second component are both a same ceramic, and wherein the first component has different material properties than the second component.

8. The end effector of claim 1, wherein the surgical device comprises a forceps, wherein the first component comprises a jaw body and the second component comprises a frame that facilitates an articulating movement of the jaw body, and wherein the one or more features of the frame comprise one or more of a pivot journal or cam interfacing slot.

9. The end effector of claim 8, wherein the joint is configured to allow a first relative movement of the first component relative to the second component during a first part of articulating movement of the first component, and wherein the joint is configured to allow a second more restrictive relative movement of the first component relative to the second component during a second part of the articulating movement of the first component.

10. The end effector of claim 1, wherein the joint has a first shape at a first portion thereof such that, during a first part of the movement of the first component, the first component is subject to a first bending moment, wherein the joint has a second shape at a second portion thereof such that during a second part of the movement of the first component the first component is subject to a second different bending moment.

11. The end effector of claim 1, wherein the joint is configured to provide for a relative movement between the first component and the second component for a first portion of the movement of the first component, and wherein the joint is configured to provide for intimate contact between the first component and the second component through a second portion of the movement of the first component.

12. The end effector of claim 1, wherein the joint is configured to provide the first component with at least two closure regimes such that a plot of at least two different bending moments through the movement has a step function.

13. The end effector of claim 1, wherein the joint has a plurality of arcuate segments each having a different degree of curvature relative to one another.

14. A forceps, comprising:
a shaft;
an actuator routed along the shaft; and
a jaw positioned at an end portion of the shaft and coupled to the actuator, the jaw comprising:
a body,
an electrode coupled to the body, and
a frame coupled to the body at a joint and coupled to the actuator, wherein, the joint is shaped such that the actuator applies at least two different forces each of a different degree to the frame through a movement of the body.

15. The forceps of claim 14, wherein the body is formed of a first electrically non-conductive material that electrically isolates the electrode, and wherein the frame is formed of a different second material with a crystalline microstructure.

16. The forceps of claim 14, wherein, when actuated by the actuator, the frame is configured to facilitate articulating movement of the body relative to the shaft, and wherein the joint is shaped to provide the body with at least two different bending moments during articulating movement of the body.

17. The forceps of claim 16, wherein the body has at least two closure regimes such that a plot of the at least two different bending moments during the articulating movement has a step function.

18. The forceps of claim 14, wherein the joint has a plurality of arcuate segments each having a different degree of curvature relative to one another, and wherein at least one of the plurality of arcuate segments is curved along an axis perpendicular to a longitudinal axis of the jaw to counteract an off-axis roll of the jaw upon initial contact with tissue of a patient.

19. The forceps of claim 14, wherein the joint allows relatively more travel of the body per an amount of applied force by the actuator upon initial contact with a tissue of a patient and through a first part of the movement, and wherein the joint allows for relatively less travel of the body with the amount of applied force by the actuator through a second part of the movement of the body.

20. An end effector for a surgical device, comprising:
a first component forming a body of the end effector; and
a second component coupled to the first component at a joint, wherein the second component connects the first component to the surgical device and has one or more features that are configured to facilitate movement of the first component, wherein the joint is shaped such that an actuator applies at least two different forces each of a different degree to the second component through an articulating movement of the body.
